(19) United States
(12) Patent Application Publication
(10) Pub. No.: US 2018/0070948 A1
(43) Pub. Date: Mar. 15, 2018

(54) SURGICAL CLIP APPLIER

(71) Applicant: Hangzhou Optcla Medical Instrument Co., Ltd., Hangzhou (CN)

(72) Inventors: Renmu TAN, Hangzhou (CN); Jin WANG, Shenzhen (CN)

(21) Appl. No.: 15/814,118

(22) Filed: Nov. 15, 2017

Related U.S. Application Data
(63) Continuation of application No. PCT/CN2015/079092, filed on May 15, 2015.

(51) Int. Cl. A61B 17/128; A61B 17/122

(52) U.S. Cl. CPC A61B 17/128; A61B 2017/00862; A61B 17/1222

(57) ABSTRACT

A surgical clip applier, including: a body provided with a trigger with an actuating state and a release state; a rod extending from the body and including a pair of jaw at the distal end away from the body; a track fixed in an inner cavity of the rod and extending to the jaw; a carrier capable of sliding along the track and connecting to the trigger, where the surgical clip is capable of being placed in the carrier; and a push plate arranged in a cavity between the carrier and the track and slidable relative to the carrier, the push plate abutting against a last clip. The push plate moves with the carrier and pushes the clip toward the jaw as the carrier moves, and the push plate doesn't retract with the carrier or stop retracting after having retracted for a pre-set distance as the carrier retracts.

### TECHNICAL FIELD

This disclosure relates to the field of medical instruments, and in particular to a surgical clip applier.

### SUMMARY

Disclosed herein are embodiments of a surgical clip applier.

### BRIEF DESCRIPTION OF THE DRAWINGS

FIG. 1 is an overall structure diagram for a surgical clip applier;

FIG. 2 is a structural schematic diagram for a clip pushing device;

FIG. 3 is a structural diagram for a track;

FIG. 4 is an assembly diagram for a carrier and a push plate;

FIG. 5 is a structural diagram for a carrier;

FIGS. 6a and 6b are two cross-sectional diagrams for a clip pushing device;

FIG. 7 is a structural diagram for a push plate;

FIG. 8 is a partial sectional diagram for a carrier, a track and a push plate that have been assembled together;

FIG. 9 is an enlarged schematic diagram for a standby magazine in FIG. 3;

FIG. 10 is a schematic diagram illustrating a first feed of a carrier;

FIG. 11 is a schematic diagram illustrating a return stroke of a carrier;

FIG. 12 is a schematic diagram illustrating a second feed of a carrier; and

FIG. 13 is an internal structure diagram for a surgical clip applier.

### DETAILED DESCRIPTION

During surgical procedures, a surgical clip may be used to ligature some blood vessels, catheters, and other channels and human tissues that require ligation, where the surgical clip may be triggered for use by a surgical clip applier. The surgical clip applier may include a housing with a trigger device and an elongated rod connected to the housing, with a jaw provided at a tail end of the rod. When in use, the jaw can be directed towards the tissue, such as blood vessels, to be clamped, the surgical clip applier can be controlled to push the surgical clip to the jaw, and then the jaw can be closed, so that a surgical clip in the jaw deforms, thereby achieving the ligation effect.

Some clip delivery mechanisms may use a spring as a drive mechanism. During the compression and release of the spring, the thrust force generated is not uniform, the delivery is too fast, and the stability is very poor. Consequently, a surgical clip may become disengaged and loosened in the delivery process or become deformed during the delivery due to compression, impact, etc., so that the clip cannot allow effective clamping or usage. During a surgical procedure, a surgical clip should be used in an accurate and timely manner, and the success of a surgery may be affected if the above problems occur. Although surgical clip appliers using other precision structures can achieve the above purpose, the clip delivery apparatuses in this case may have a complex internal mechanical structure, in which a push rod for feeding is made of plastics and has high requirements on its dimensional accuracy, wear resistance and lubrication performance. It is difficult to meet these requirements, thereby resulting in high cost, and accordingly, the clip delivery apparatuses currently manufactured cannot meet the market demand.

The present disclosure includes a surgical clip applier which can provide timely and smooth clip delivery without clip stuck or clip deformation, and can be relatively simple in structure and low in manufacturing cost.

In one embodiment, a surgical clip applier may include a body, a rod extending from the body, a track, a carrier sliding along the track and a push plate.

The body may include a trigger device that may be provided with an actuated state and a released state.

The rod may be hollow and be provided with an inner cavity formed therein, and a jaw for bending a surgical clip may be provided at a distal end far from the body.

The track may be fixed in the inner cavity of the rod and extend to the jaw along an axial direction of the rod.

The carrier may be capable of accommodating at least one surgical clip therein. The carrier may be connected to the trigger device, such that the carrier may be capable of moving toward the jaw along the track when the trigger device is actuated, and the carrier may be capable of moving toward the body along the track when the trigger device is released.

The push plate may be arranged in a cavity formed between the carrier and the track and slidably fitted to the carrier. The push plate may be capable of moving along with the carrier and pushing a surgical clip toward the jaw when the carrier moves toward the jaw along the track, and the push plate may not be capable of retracting along with the carrier or be capable of retracting for less than a predetermined distance when the carrier retracts toward the body along the track.

In one embodiment, the carrier is connected to the trigger device; when the trigger device is switched to a triggered position, the carrier can move toward the jaw along the track and drive the push plate to move toward the jaw, and the push plate can push a surgical clip toward the jaw while moving along with the carrier; and when the trigger device is switched to a released position, the carrier can move toward the body along the track, and meanwhile, the push plate may not retract along with the carrier toward the body, thus avoiding the problem that a surgical clip gets stuck and deformed due to being pressed and improving the stability of the applier in clip delivery.

A clip pushing device and a surgical clip applier may be provided as described further in detail below. It can be understood that the embodiments described herein are only intended to explain this disclosure rather than limiting this disclosure.

With reference to FIG. 1, a surgical clip applier may include a body 10, a rod 20, a jaw 30, and a clip pushing device. The body 10 may include a housing 13 and a trigger device for driving the clip pushing device. The trigger device is movably connected to the body 10. The trigger device may be provided with an actuated state and a released state. The trigger device may include a handle 12, and the trigger device can be switched into the actuated state by operating the handle 12. The rod 20 may extend from the housing 13 of the body 10. The body 10 may define an axial direction, and the rod 20 can extend distally in the axial direction. The rod 20 may be hollow and arranged with the jaw 30 for bending a surgical clip at its distal end away from the body. The clip pushing device and surgical clip(s) may be received inside a hollow cavity of the rod 20 (hereinafter referred to as an inner cavity of the rod). The clip pushing device may be coupled to the trigger device, and through the action of the trigger device, the clip pushing device can push a surgical clip to the jaw 30. In an exemplary embodiment, when the clip applier is in an unused state, the trigger device may be in the released state, and the clip pushing device may be in a to-be-pushed state. When the handle 12 is operated to get the trigger device into the actuated state, the trigger device may apply a pushing force to the clip pushing device, so that the clip pushing device can push a surgical clip toward the jaw 30. When the handle 12 is released, the trigger device may return to the released state to apply a pulling force to the clip pushing device, such that the clip pushing device may return to the to-be-pushed state.

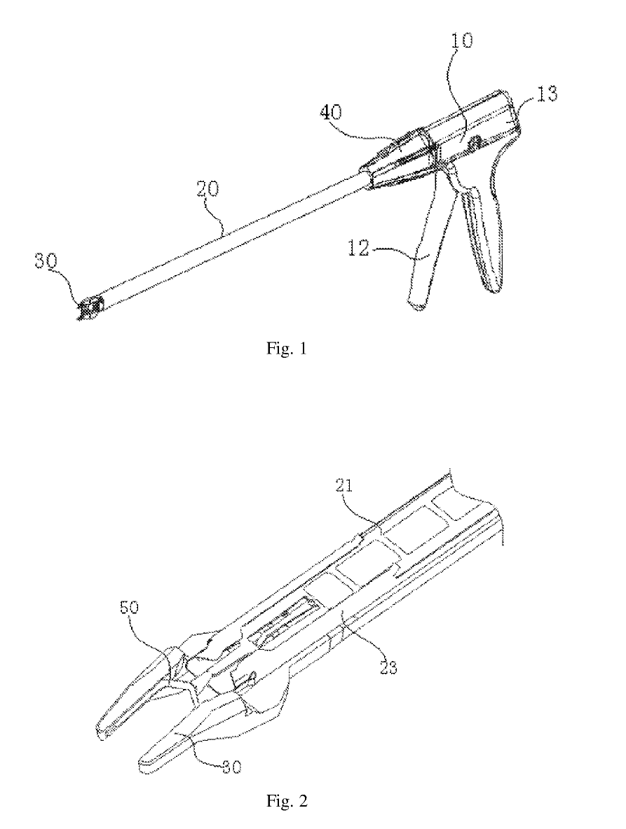

With reference to FIGS. 2-8, the clip pushing device may include a track 23, a carrier 21, and a push plate 22. The track 23 may be fixed within the inner cavity in the axial direction of the rod; for example, the track 23 can be fixed to a wall of the rod. The track 23 may be in an elongated shape and extend to the jaw 30. The track 23 may include a locating mechanism, which is used for restricting the carrier 21 to slide along the track 30 and maintaining a distance between the carrier 21 and the track 23. In one embodiment, the locating mechanism may be side wall(s) of the track 23.

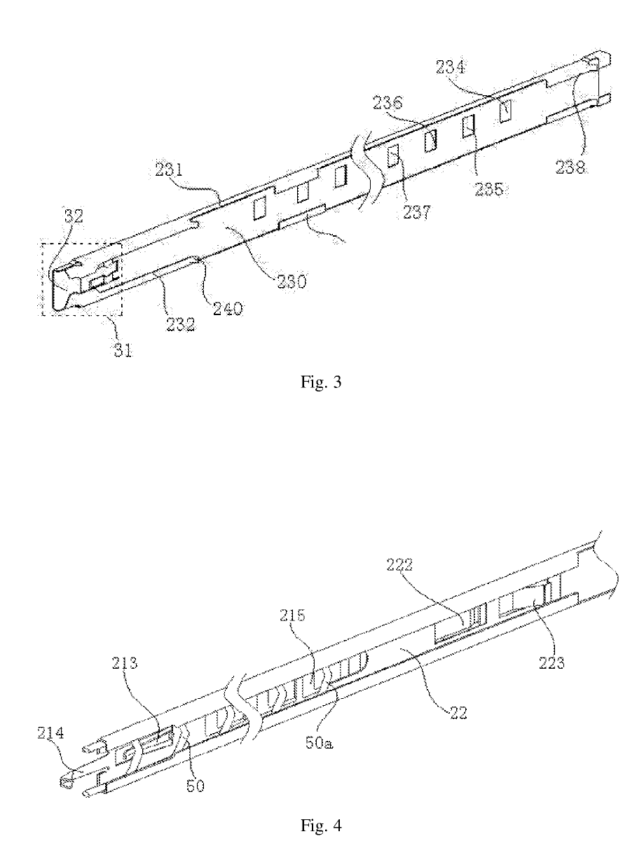

As shown in FIG. 3, the track 23 may include a first bottom surface 230 extending in a lengthwise direction of the track and two first side walls 231 perpendicular to the first bottom surface 230, where the first bottom surface 230 and the two first side walls 231 may define a U-shaped groove. Here, the U-shaped groove may mean that a cross-section of the groove is U-shaped, and the first bottom surface of the groove can either be curved or planar. In one embodiment, the first bottom surface 230 may be planar, and the locating mechanism may refer to the side walls and folded edges of the side walls of the track. In one embodiment, at least a portion of the two first side walls 231 of the track may be provided with inwardly bent folded edges, which can be used to form a position restriction for the carrier 21 located in the track 23 so as to prevent the carrier 21 from being disengaged from the track 23.

As further shown in FIG. 3, the two first side walls 231 at two ends of the track 23 may include inwardly bent folded edges 238, and similar folded edges 238 can also be arranged at several positions in the middle portion of the track. In one embodiment, the track may include no side wall, and the locating mechanism may be several “L”-shaped structural members extending perpendicularly from the first bottom surface of the track. The “L”-shaped structural member may include a first rod and a second rod which two are substantially perpendicularly connected together at one end. A free end of the first rod of the “L”-shaped structural member may be connected to the first bottom surface, and the second rod of the “L”-shaped structural member may provide position restriction for the carrier 21.

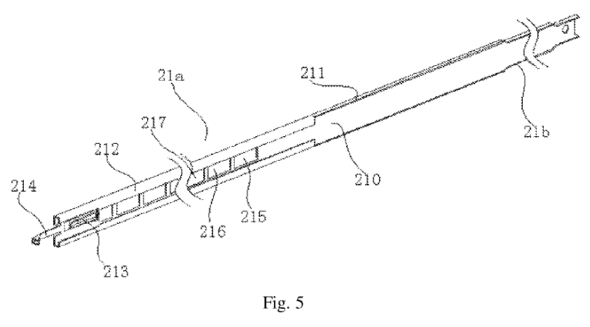

The carrier 21 may cooperate with the locating mechanism and slide along the track under a constraint of the locating mechanism. As shown in FIGS. 4 and 5, the carrier 21 may include a second bottom surface 210 extending in a lengthwise direction of the carrier 21 and two second side walls 211 perpendicular to the second bottom surface 210. The second bottom surface 210 and the two second side walls 211 may also define a U-shaped groove. At least one surgical clip 50, for example, a string of surgical clips, can be placed inside the carrier. The push plate 22 may be arranged on the carrier and slidably fitted to the carrier, and the push plate 22 may abut against a last surgical clip 50a accommodated inside the carrier 21. In one embodiment, at least a section of the two second side walls of the carrier 21, which section is located close to the jaw, may be provided with inward folded edges, and the second bottom surface and the second side walls of the carrier and the folded edges of the second side walls can define a semi-closed groove for receiving clip(s) and the push plate, so as to prevent the clip(s) and the push plate from being disengaged from the carrier. In the embodiment as shown in FIG. 5, the second side walls 211 at the end of the carrier 21, which end is close to the jaw 30, can be bent inwardly to obtain the folded edges 212, so that the second bottom surface 210, the second side walls 211 and the folded edges 212 in this section can define a storage area 21a of the semi-closed groove, where the storage area 21a can be used for holding a set of clips 50 in sequence. The carrier 21 may be provided, at an end thereof away from the jaw 30, with an assembly area 21b for connection to the trigger device.

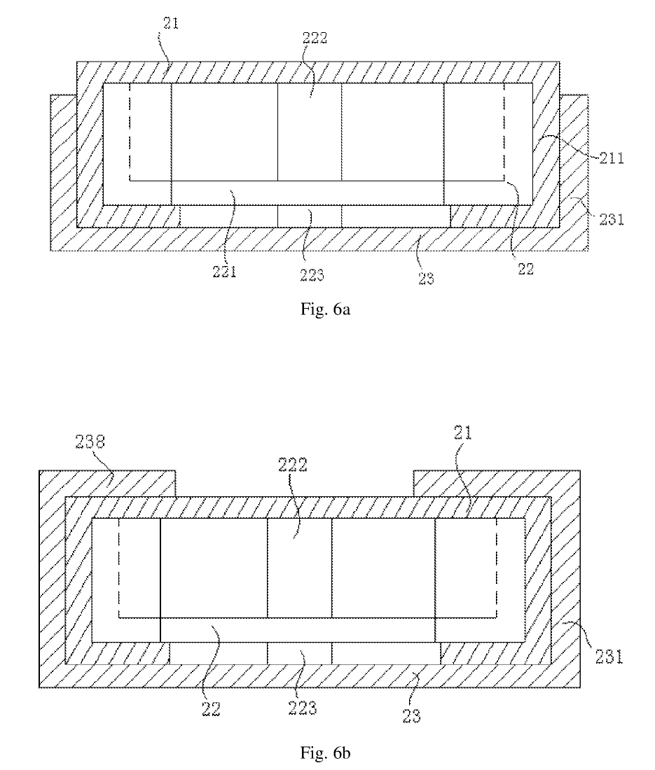

FIG. 6a is a cross-sectional diagram in which the locating mechanism of the track is the first side walls of the track. The carrier 21 and the track 23 may be snap fitted together with their respective openings facing each other, in which case the two second side walls 211 of the carrier 21 can be located on an inner side of the two first side walls 231 of the track 23, and the push plate 22 can be located inside a cavity formed by the carrier 21 and the track 23 in sleeve connection with each other. The width of the carrier 21 is slightly less than that of the track 22, so as to ensure that the groove formed by the carrier 21 can slide within the groove formed by the track 23. In some cases, the width of the carrier and the track can make sure that the second side walls 211 of the carrier 21 and the first side walls 231 of the track 23 may not be loosen while sliding with respect to each other. In one embodiment, the carrier 21 can be in clearance fit with the track 23.

FIG. 6b is a cross-sectional schematic diagram in which the locating mechanism of the track is either the first side walls of the track and the folded edges of the first side walls or the first side walls of the track and the “L”-shaped structural member. In one embodiment, the carrier may be connected to the trigger device, so that when the trigger device is actuated, the carrier may move toward the jaw along the track and drive the push plate to move toward the jaw, and the push plate may push a surgical clip toward the jaw while moving along with the carrier; and when the trigger device is released, the carrier may move toward the body along the track, and meanwhile, the push plate may not retract along with the carrier or stop retracting after having retracted for less than a predetermined distance.

In one embodiment, the push plate 22 may be provided with a first locating protrusion projecting toward the track 23 and a second locating protrusion projecting toward the carrier 21. The track may be provided axially with a plurality of retaining walls cooperating with the first locating protrusion. The first locating protrusion may abut against the retaining walls so as to prevent the push plate from sliding toward the body. The carrier may be provided axially with a plurality of push walls, and when the carrier moves toward the jaw, one of the push walls can abut against the second locating protrusion to drive the push plate to move toward the jaw. In one embodiment, the push plate 22 may be provided with a first locating elastic piece 222 as the first locating protrusion and a second locating elastic piece 223 as the second locating protrusion. Fixed ends of the first locating elastic piece 222 and the second locating elastic piece 223 may be located closer to the jaw than free ends thereof are, and the free ends of the first locating elastic piece 222 and the second locating elastic piece 223 may tilt toward the track 23 and the carrier 21, respectively. The track 23 and the carrier 21 can be provided with locating recesses functioning as the retaining walls and the push walls, so as to be adapted to the first locating elastic piece 222 and the second locating elastic piece 223, respectively. The locating recesses and the locating elastic pieces may form ratchet structures. In one embodiment, as shown in FIG. 3, the locating recesses on the track 23 may be holes 234, 235, 236 and 237. As shown in FIG. 5, the locating recesses of the carrier 21 may be grooves 215, 216 and 217. Alternatively, the locating recesses on the track 23 may be grooves, and the locating recesses of the carriers 21 may be holes. The plurality of locating recesses can be uniformly spaced on the carrier 21 and the track 23 with the spacing between those locating recesses being equal to the spacing between clips 50, and the number of the locating recesses is greater than that of the clips 50 in the carrier 21.

Using the surgical clip applier of this disclosure, when the applier is not operated, the clip pushing device is in the to-be-pushed state; when the trigger device is actuated, the trigger device may push the carrier 21 to move toward the jaw 30 along the track 23; and when the carrier 21 is engaged with the push plate 22 therein by means of its locating recesses, the push plate 22 may be pushed to move toward the jaw 30, while a clip 50 inside the carrier 21 may also move toward the jaw under the pushing action of the push plate 22. When the trigger device is released, the trigger device may pull the carrier 21 to move toward the body along the track 23 to cause the carrier 21 to return. However, since the ratchet structure formed by the locating recesses on the track 23 and the locating protrusion on the push plate 22 restricts movement of the push plate 22 to be a unidirectional movement toward the jaw 30, the push plate 22 may remain in the position after its movement toward the jaw, instead of returning synchronously with the carrier 21, or, the push plate 22 may first retract along with the carrier for less than a predetermined distance under the action of the frictional force between the carrier 21 and the push plate 22 and then the push plate 22 may be stopped from further retraction due to the engagement of the locating recesses on the track 23 with the locating protrusion on the push plate 22, where the predetermined distance may refer to the spacing between two locating recesses on the track 23. Through the above operation, due to the pushing action of the push plate 22, a clip 50 in the carrier 21 can advance a distance of one time of feed relative to the original position. As such, after multiple times of feeds of the carrier 21, clips 50 in the carrier 21 can be pushed toward the jaw 30 one by one.

It may be understood by those skilled in the art that the unidirectional movement of the push plate can also be achieved by other structures according to this disclosure.

Compared with a spring-type clip delivery mechanism, a ratchet structure is adopted to prevent a surgical clip from retraction in a reverse direction toward the body, so that a surgical clip can only move uni-directionally toward the jaw, thereby preventing a surgical clip from getting stuck and deformed due to being pressed and realizing stable clip delivery for the applier.

In one embodiment, the track can be fixedly arranged, and the carrier can be reliably pressed close to the track by the locating mechanism, which on one hand can ensure the carrier to slide along the track, and on the other hand can avoid changes in the distance between the push plate and the carrier as well as changes in the distance between the push plate and the track, so that the locating elastic pieces on the push plate can be reliably pressed into the locating recesses on the carrier and on the track respectively, thereby improving the reliability of the clip delivery of the clip pushing device. In addition, such cooperation among the carrier, the push plate and the track may be achieved based on relatively easy manufacture, and the carrier and the track can be made of metal material, so that it is possible to achieve the dimensional accuracy and miniaturization, and it is also possible to meet the requirements on the wear resistance and lubrication performance.

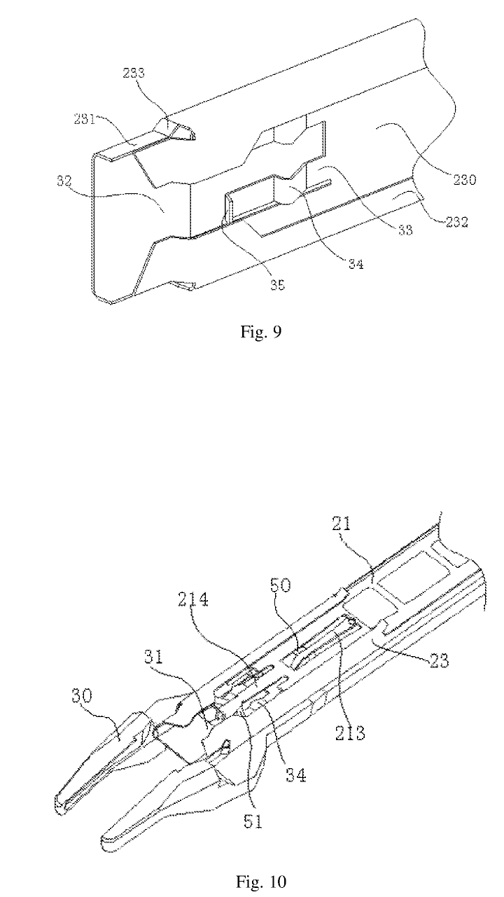

In one embodiment, the end of the track engaged with the jaw may be further provided with a standby magazine for fixing clip(s). As shown in FIGS. 3 and 9, free ends of the first side walls 231 at the end of the track 23 that is close to the jaw 30 may be bent inwardly to form the folded edges 232, and the first bottom surface 230, the first side walls 231 and the folded edges 232 of this section of the track can define a standby magazine 31, where the standby magazine 31 may be a semi-closed groove. The semi-closed groove of the standby magazine may extend backward (i.e., toward the body) for a distance to obtain an extension section, and the folded edges 232 at a rear end of the extension section may tilt away from the first bottom surface 230 to form a guide member 240, so as to facilitate a front end of the carrier 21 to slide into the standby magazine 31. For clarity, it is defined herein that a direction toward the jaw along the axial direction of the rod is a forward/front direction, and a direction toward the body along the axial direction of the rod is a rearward direction. Accordingly, when a first object is located closer to the jaw than a second object is, it may be considered that the first object is located in front of the second object, and correspondingly, the second object is located behind the first object.

In order to locate a surgical clip in the standby magazine to avoid undesired movement of the clip, the first bottom surface of the standby magazine 31 may be provided with a first stop member for restricting the movement of a clip in the standby chamber. As shown in FIGS. 3 and 9, one or more elastic pieces 33 may be provided on the first bottom surface 230 at the rear end of semi-closed section of the standby magazine 31. A base portion of each elastic piece 33, which base portion is located away from the jaw 30, may protrude away from the first bottom surface 230 so as to form a rear stop member 34, where the rear stop member 34 may be located behind a clip in the standby magazine 31 to prevent a clip from moving toward the body. In an exemplary embodiment, the rear stop member 34 may be a tapered protrusion having different slope on its two sides, which two sides may include a first side facing away from the jaw and a second side facing the jaw. The first side may be an inclined surface at a certain angle with respect to the first bottom surface 230, and the angle may be designed to facilitate a clip 50 to slide over the rear stop member 34. The second side may be a vertical surface perpendicular to the first bottom surface 230 or an inclined surface at a slightly greater angle with respect to the first bottom surface 230, i.e., the slope of the second side is greater than that of the first side, thereby preventing a clip 50 from moving away from the jaw 30 (i.e., toward the body).

In order to further ensure that a clip 50 may not have undesired movement in the standby magazine 31, an end of the elastic piece 33, which end is located toward the jaw, may be bent away from the first bottom surface 230 to form a front stop member 35, and a bending angle of the front stop member 35 may be set in such a way that a clip 50 cannot pass the front stop member 35 without an external force applied. In one embodiment, the bending angle can be set to be an acute angle.

In one embodiment, the first stop member may include the front stop member 35 and the rear stop member 34, and in other embodiments, the front stop member 35 or the rear stop member 34 may be alternatively provided. The front stop member may be located in front of a clip in the standby magazine, and thus the front stop member can be used for preventing a clip from moving toward the jaw in an uncompressed state (i.e., unoperated/released state of the trigger device) and for allowing a clip to pass through the front stop member and move toward the jaw in a compressed state (i.e., the actuated state of the trigger device). The rear stop member may be located behind a clip in the standby magazine and used for preventing a clip from moving toward the body.

In one embodiment, the front end of the standby magazine 31 may be provided with a neck 32 which may taper in the cross-section toward the jaw. As shown in FIGS. 3 and 9, the first side walls 231 may bend inwardly in a stepped manner to form the neck 32, and a convex structure 233 for reinforcing the elastic force may be provided at the portion of the first side walls from which the first side walls start to bend inwardly stepwise. The width of the cross-section of the neck 32 may be less than the size of a clip 50, so that a clip 50 cannot pass through the neck 32 without an external force applied, thereby limiting the displacement of a clip 50 toward the jaw.

In other embodiments, a plurality of elastic stop pieces 213 may be provided, so as to ensure that a clip 50 cannot move toward the jaw 30 without operation control.

As shown in FIGS. 4 and 5, a push head 214 may be provided at the end of the carrier 21 toward the jaw 30, where the push head 214 may extend from an end surface of the second bottom surface 210 of the carrier 21 toward the jaw 30 and bend toward an inner side of the cavity to such an extent that an end of the push head can be in close contact with the first bottom surface 230 of the track 23 when assembled. The end of the push head 214 may be provided with a groove adapted to the shape of a rear section of a clip 50 so as to push the clip 50 conveniently. When the carrier moves toward the jaw, the push head may be capable of stretching into the standby magazine to push a surgical clip in the standby magazine into the jaw. As shown in FIG. 3, In one embodiment, a set of elastic pieces 33 in the standby magazine 31 may be arranged opposite each other close to the first side walls 231, respectively, and a gap can be provided between the set of elastic pieces 33 so that the push head 214 may pass through the gap and push a clip 50 into the jaw 30.

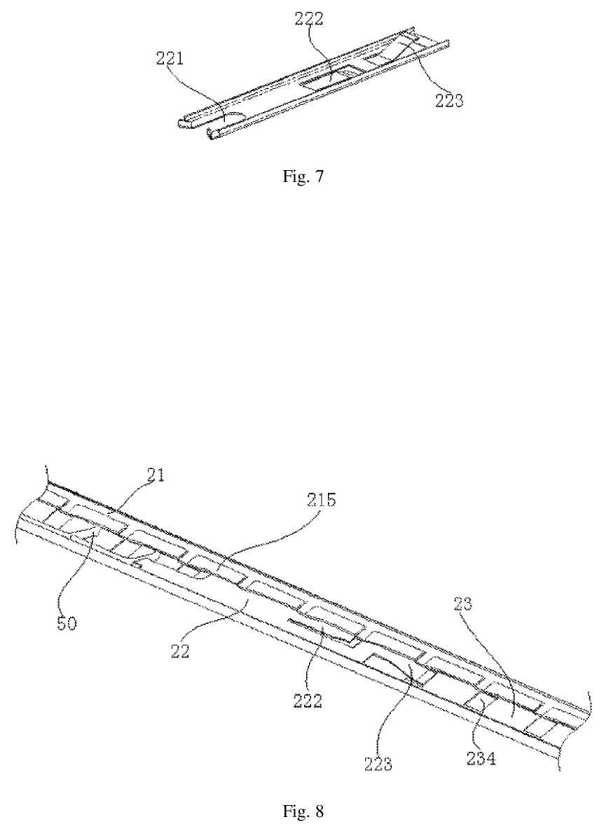

As shown in FIG. 7, a notch 221 adapted to the push head 214 may be provided on the side of the push plate 22 toward the jaw 30 so as to facilitate the push head 214 to push a last clip 50 into the jaw 30.

Further, as shown in FIGS. 4 and 5, the carrier 21 may be further provided with a second stop member that may be located behind the push head 214 and used for preventing clip(s) outside the standby magazine from moving toward the jaw. In one embodiment, the second stop member may be the elastic stop piece 213 extending from the second bottom surface 210 of the carrier and extending toward the jaw in the axial direction, and the end of the elastic stop piece 213 may be provided with a hook-like structure facing the track for limiting the displacement of a clip 50 outside the standby magazine 31 toward the jaw 30, so that a clip 50 cannot move toward the jaw 30 without active control. It is mentioned that the hook-like structure at the end of the elastic stop piece 213 may use a structure with a bending angle no more than 90° so as to avoid the problem of high resistance force during feeding process due to too large bending angle.

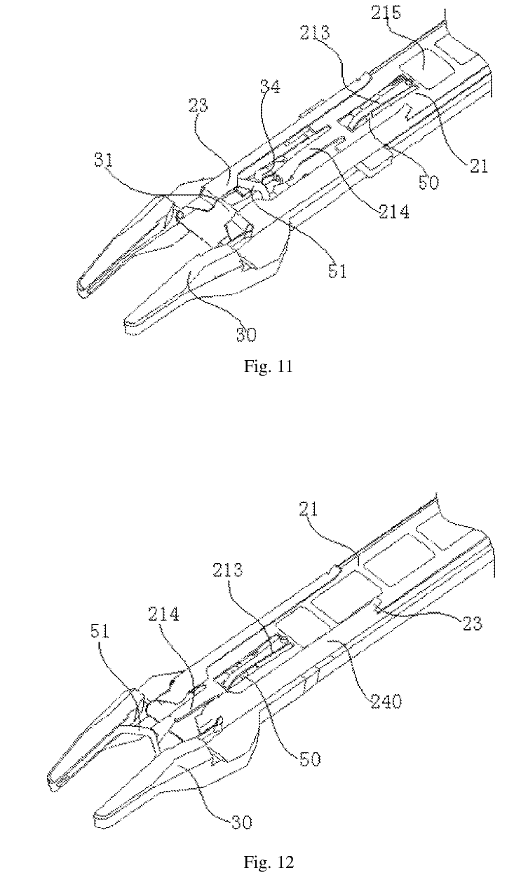

FIGS. 10-12 illustrate the pushing process of a clip. According to the distance from the jaw, from the rear direction to the front direction, the clips can be marked as 50, 51, . . . , the grooves on the carrier 21 can be successively marked as 215, 216, 217, 218, . . . , and the holes in the track 23 can be successively marked as 234, 235, 236, 237, . . . . As shown in FIG. 10, when a clip 51 enters the standby magazine 31, the locating elastic piece 222 may be located within the groove 215, and the locating elastic piece 223 may be located between the holes 235 and 236. Then the carrier 21 returns, as shown in FIG. 11, the elastic stop piece 213 may push a clip 50 to return, and the clip 50 pushes the push plate 22 in the rear direction (i.e., toward the body) until the locating elastic pieces 222, 223 enter the groove 216 and the hole 235. As shown in FIG. 12, when the carrier 21 is fed again, the clip 51 may be pushed into the jaw 30 by the push head 214, and the push plate 22 may simultaneously push the clip 50 into the standby magazine 31 by compressing the front stop member 35. At this point, the locating elastic piece 222 may be located within the groove 216, the locating elastic piece 223 may be located between the holes 236 and 237, and then the carrier 21 may start to return again. Such operation may be repeated such that each time the feed distance of the push head 214 can exceed the push plate 22 by a distance, thereby ensuring the clip 50 to be pushed into the jaw 30.

In the pushing process described above, when the push plate 22 pushes the clip 51 into the standby magazine 31, the push head 214 may be located in front of the clip 51, and the clip 51 cannot slide freely toward the jaw 30 due to the limitation of the front stop member 35; and when the push head 214 returns, the push head may cross over the clip 51 and become located behind the clip 51, and due to the limitation of the rear stop member 34, the clip 51 may stay at a position between the front stop member 35 and the rear stop member 34 instead of being brought back by the push head 214. When the carrier 21 is fed again, the push head 214 can push the clip 51 located in the standby magazine 31 into the jaw 30, while the push plate 22 can push the clip 50 into the standby magazine 31.

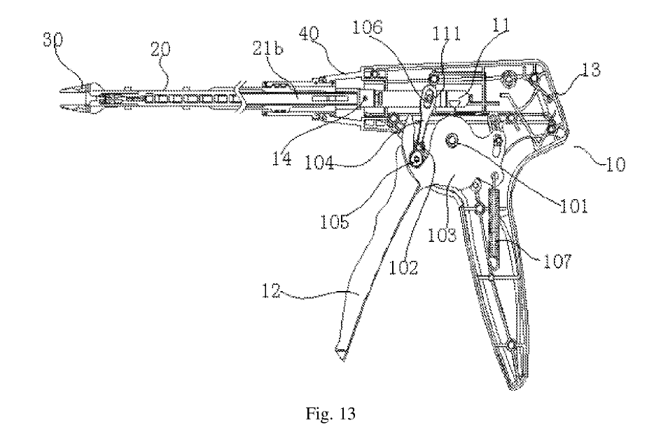

It is described below that how the trigger device may control the carrier to move. As shown in FIG. 13, the body 10 may include: the housing 13; a handle 12; a trigger transition device 103 fixedly connected to the handle 12 and rotatably arranged about a first shaft 101 on the housing 13; and a clip delivery lever 104 rotatable about a second shaft 102 fixed to the housing 13. One end of the clip delivery lever 104 may be provided with a rotary wheel 105, and the other end thereof may be provided with a sliding groove 106. A push rod 11 for transmission may be further provided between the trigger device and the pushing device. The push rod 11 can be made of a material which is easy for high precision manufacture and is resistant to friction. In one embodiment, the push rod 11 can be made of metal material. The push rod 11 may be provided with a pin 111, and the pin 111 may pass through the sliding groove 106 and is slidable therein. A front end of the trigger transition member 103 may be provided with an arc groove, which may abut against the rotary wheel 105 and drive the clip delivery lever 104 to rotate about the second shaft 102. When the handle 12 is operated to be opened or closed, the trigger transition member 103 on the handle 12 and the rotary wheel 105 on the clip delivery lever 104 may form a cam pair to drive the clip delivery lever 104 to rotate, so that the pin 111 can slide in the sliding groove 106. The push rod 11 is restricted in the vertical displacement by other structures, so the pin 111 can only move laterally when sliding in the sliding groove 106, thereby driving the pushing rod 11 to move back and forth. The push rod 11 may drive, by moving back and forth, a push assembly 200 to generate feed and return motions. In this way, when the handle 12 is operated to be closed toward the housing 13, the trigger transition member 103 on the handle 12 and the rotary wheel 105 on the clip delivery lever 104 may form a cam pair to drive the bottom of the clip delivery lever 104 to rotate counter-clockwise, while the pin 111 may drive the push rod 11 to move toward the jaw 30, and the pushing device may be driven by the push rod 11 to perform the feed motion.

Further, a spring 107 fixing to the housing 13 may exert a pulling force on a rear end of the trigger transition member 103. When the handle 12 is released, the push rod 11 may return under the action of the pulling force of the spring 107 to drive the pushing device to return.

The push rod 11 may be connected to the assembly area 21b of the carrier 21 through a coupler 14, and a rotary handle 40 fixedly connected to the coupler 14 may be provided between the rod 20 and the body 10. By rotating the rotary handle 40, the carrier 21 and the track 23 can drive the jaw 30 to rotate so as to adjust the position of the jaw.

The surgical clip applier may also include a control assembly which can control the closing or opening motion of the jaw 30 by means of the closing and opening of the handle 13. The control assembly may be connected to the trigger device in the body 10 by means of a cam device at the jaw 30 through a connection rod, thereby achieving the control operation of the jaw 30 which is synchronous with the feeding of the clip pushing device. The control assembly has been known for those skilled in the art and will not be described here again.

In the surgical clip pushing device and the surgical clip applier provided in the above embodiments, the unidirectional feeding mode of a ratchet type between the track, the push plate and the carrier is used, the standby magazine for stabilizing a to-be-triggered clip is provided at the jaw, and a metal push rod easy to be processed is used, so that it not only can realize stable clip delivery, but also the surgical clip may not get deformed or stuck, and moreover, as the push rod is made of metal, the technological requirements are greatly lowered, and costs can be reduced.

This disclosure has been described in detail with reference to specific examples, which are merely for the purpose of facilitating understanding of this disclosure and are not intended to limit this disclosure. It will be apparent to those skilled in the art that changes may be made to the specific embodiments described above in accordance with the teachings of the present invention.

1. A surgical clip applier, comprising:
a body that comprises a trigger device, wherein the trigger device is provided with an actuated state and a released state;
a rod that extends from the body, wherein the rod is hollow and comprises an inner cavity therein; wherein a jaw is provided at a distal end of the rod away from the body for bending a surgical clip;
a track that is fixed in the inner cavity of the rod and extends to the jaw in an axial direction of the rod;
a carrier that is capable of sliding along the track and accommodating at least one surgical clip therein; wherein the carrier is connected to the trigger device, wherein the carrier is capable of moving toward the jaw along the track when the trigger device is actuated, and wherein the carrier is capable of moving toward the body along the track when the trigger device is released; and
a push plate that is arranged in a cavity formed between the carrier and the track and slidably fitted to the carrier; wherein the push plate is capable of moving along with the carrier and pushing a surgical clip toward the jaw when the carrier moves toward the jaw along the track, and wherein the push plate is incapable of retracting along with the carrier or is capable of retracting for less than a predetermined distance when the carrier retracts toward the body along the track.

2. The surgical clip applier of claim 1, wherein the track comprises a locating mechanism, and the carrier cooperates with the locating mechanism to slide along the track under a constraint of the locating mechanism.

3. The surgical clip applier of claim 2, wherein the carrier comprises a second U-shaped groove which is formed by a second bottom surface extending in a lengthwise direction of the carrier and two second side walls substantially perpendicular to the second bottom surface, and the second U-shaped groove is capable of holding the push plate and a surgical clip.

4. The surgical clip applier of claim 3, wherein the track comprises a first U-shaped groove which is formed by a first bottom surface extending in a lengthwise direction of the track and two first side walls substantially perpendicular to the first bottom surface; wherein the carrier and the track are snap fitted together with respective openings facing each other, and wherein the two second side walls of the carrier are located inside the two first side walls of the track.

5. The surgical clip applier of claim 4, wherein at least a portion of the two first side walls of the track comprises first folded edges that are bent inwardly to form a position limit for the carrier.

6. The surgical clip applier of claim 3, wherein at least a section of the two second side walls of the carrier, which section is located toward the jaw, comprises second folded edges that are bent inwardly, and the second bottom surface, the second side walls and the second folded edges of the carrier form a semi-closed groove for receiving a surgical clip and the push plate.

7. The surgical clip applier of claim 1, wherein a distal end of the track is provided with a standby magazine for holding a surgical clip, and the standby magazine comprises a semi-closed groove for receiving a surgical clip.

8. The surgical clip applier of claim 7, wherein the standby magazine comprises a first bottom surface and two first side walls substantially perpendicular to the first bottom surface, wherein the first side walls are bent inwardly to form first folded edges which constitute the semi-closed groove together with the first bottom surface and the first side walls, and wherein the first bottom surface is provided with a first stop member for preventing movement of a surgical clip inside the standby magazine.

9. The surgical clip applier of claim 8, wherein the first stop member comprises a rear stop member that is located behind a surgical clip in the standby magazine and is used for preventing a surgical clip from moving toward the body.

10. The surgical clip applier of claim 8, wherein the first stop member comprises a front stop member located in front of a surgical clip in the standby magazine; wherein the front stop member is used for preventing a surgical clip from moving toward the jaw in an uncompressed state and for allowing a surgical clip to pass through the front stop member and move toward the jaw in a compressed state.

11. The surgical clip applier of claim 7, wherein one end of the standby magazine, which end is engaged with the jaw, is provided with a neck with a cross-section tapering toward the jaw, and a cross-sectional dimension of the neck in at least one direction is less than that of a surgical clip.

12. The surgical clip applier of claim 11, wherein one end of one of the two first side walls of the standby magazine that is located close to the jaw and one end of the other one of the two first side walls of the standby magazine that is located close to the jaw are capable of elastically deforming toward each other to form the neck.

13. The surgical clip applier of claim 7, wherein one end of the carrier, which end is located close to the standby magazine, is provided with a push head extending toward the jaw, and when the carrier moves toward the jaw, the push head is capable of extending into the standby magazine to push a surgical clip in the standby magazine into the jaw.

14. The surgical clip applier of claim 13, wherein the carrier is further provided with at least one second stop member behind the push head for preventing a surgical clip outside the standby magazine from moving toward the jaw.

15. The surgical clip applier of claim 14, wherein the second stop member is an elastic stop piece which extends from a second bottom surface of the carrier and extends toward the jaw, and an end of the elastic stop piece is provided with a hook-like structure facing the track.

16. The surgical clip applier of claim 1, wherein the push plate comprises a first locating protrusion projecting toward the track and a second locating protrusion projecting toward the carrier; the track is provided axially with a plurality of retaining walls cooperating with the first locating protrusion, and the first locating protrusion is capable of abutting against one of the retaining walls to prevent the push plate from sliding toward the body; the carrier is provided axially with a plurality of push walls, and when the carrier moves toward the jaw, one of the push walls is capable of abutting against the second locating protrusion to push the push plate to move toward the jaw.

17. The surgical clip applier of claim 1, wherein the carrier and the track are made of metal material.

18. The surgical clip applier of claim 1, further comprising a push rod which is connected to the trigger device for feeding the carrier, wherein the push rod is a metal push rod.
